(19) United States
(12) Patent Application Publication
(10) Pub. No.: US 2025/0143906 A1
(43) Pub. Date: May 8, 2025

(54) DEVICES AND METHODS TO MITIGATE PATELLA MALPOSITIONING

(71) Applicant: Jill C. POKASKI AZAR, Canton, MA (US)

(72) Inventor: Jill C. POKASKI AZAR, Canton, MA (US)

(21) Appl. No.: 18/387,842

(22) Filed: Nov. 7, 2023

(51) Int. Cl. A61F 5/01

(52) U.S. Cl. CPC A61F 5/0109; A61F 2005/0197

(57) ABSTRACT

Apparatus and methods of use are provided. A patella anchor includes a firm rod member that is dimensioned and configured to trace and apply sideways pressure to a perimeter edge of a human patella in a direction parallel to a central plane of the patella. The pressure is applied via portions of tissue covering and proximate to the same perimeter edge. The perimeter edge may include a lateral, medial, top, or bottom edge of the patella. The patella anchor may further include a firm pad dimensioned and configured to apply posterior pressure to a central anterior portion of the patella in a direction toward a posterior position in relation to the anterior surface of the patella. The posterior pressure is applied via portions of the tissue covering the central anterior portion of the patella. A carrying web is provided which is configured to carry the patella anchor. At least one controlling member is provided, which is configured to apply at least one mitigating force vector to the patella anchor. The controlling member or members may include a push type of support configured to push the patella anchor into the perimeter edge. The controlling member or members may comprise a pull type of support configured to support a controlling member configured to pull the patella anchor into the perimeter edge.

### FIELD OF THE DISCLOSURE

Aspects of the disclosure relate to mitigating drift, instability, and/or malpositioning of the human patella.

### BACKGROUND

Various devices have been proposed to brace human knee structures, for example, as disclosed in U.S. Pat. Nos. 5,711,312, 4,466,428, US 2019/0328580 (see, e.g., FIGS. 8-9 and Example 5), U.S. Pat. No. 11,337,840, and US 2023/0201017.

Medical professionals and researchers have investigated the relative merits of and indications for employing bracing and patellar taping to treat patellar issues. For example, S J Warden et al. authored a paper describing their systematic review and meta-analysis about patellar taping and bracing for the treatment of chronic knee pain. S J Warden et al., “Patellar Taping and Bracing for the Treatment of Chronic Knee Pain: A Systematic Review and Meta-analysis,” Database of Abstracts of Reviews of Effects (DARE): Quality-Assessed Reviews [Internet], York (UK), Database entry date Mar. 31, 2009, pp. 1-4. The authors concluded that medially directed tape reduced chronic pain, but that there was limited evidence for patellar bracing.

Engy F. Adley et al. authored a study involving a comparison between the effect of McConnell taping, kinesiology taping, and open knee bracing in the treatment of patellofemoral pain. The study concluded that McConnell taping was more effective than the other modalities in improving pain and Kujula score, but not for knee flexion in the active range of motion. Med. J. Cairo Univ., Vol. 89, No. 5, September: 1889-1898, 2021.

Naoko Aminaka et al. authored an article entitled “Patellar Taping, Patellofemoral Pain Syndrome, Lower Extremity Kinematics, and Dynamic Postural Control.” The purpose of the study was to evaluate the effects of patellar taping on sagittal plane hip and knee kinematics, reach distance, and perceived pain level during the Star Excursion Balance Test in individuals with and without patellofemoral pain syndrome (PFPS). The article concludes that patellar taping seems to alleviate pain and improve performance on the Star Excursion Balance Test in participants with PFPS, but states that the underlying mechanisms are still unclear. The authors report that significant kinematic alterations were not found. They also report that patellar taping seems to be a safe and effective way to reduce the painful symptoms of PFPS, and may be beneficial in allowing patients to engage in functional rehabilitation exercises. Journal of Athletic Training 43(1) (2008), pages 21-28.

Teddy Worrell et al. describe in an article entitled “Effect on Patellar Taping and Bracing on Patellar Position as Determined by MRI in Patients with Patellofemoral Pain,” a study to determine the effects of patellar taping, bracing, and not taping on patellar position. The study concluded that patellar bracing and taping influenced patellar position, specifically patellofemoral congruence angle and lateral patellar displacement at ten degrees of knee flexion during a static MRI condition. More specifically, the study concluded that subjects with “normal” patellar alignment decreased their lateral patellar displacement while wearing the brace and decreased their patellofemoral congruence angle at 10 degrees of flexion while wearing tape and brace.

Scott F. Dye, MD explains in the abstract to his 2001 paper entitled “Patellofemoral Pain: A Current Perspective” that several “theories have been proposed to clarify the common problem of patellofemoral pain, but none has been fully proved. A new theory holds that restoration of tissue homeostasis, or normal metabolic function at the molecular and cellular levels, is more important for symptom resolution than the presence of cartilage damage or patellofemoral alignment characteristics. Assessment of patients who present with complaints of patellofemoral pain relies on the history and physical examination more than on imaging studies. Non-operative treatment, aimed at restoration of lost tissue homeostasis and associated resolution to a pain-free condition, includes temporary load restriction, anti-inflammatory therapy, and rehabilitation. If that does not succeed, a cautious approach to surgery may be warranted.” Dye, Scott F. “Patellofemoral pain: A current perspective.” The Journal of Musculoskeletal Medicine, vol. 18, no. 9, September 2001, p. 440. Gale OneFile: Health and Medicine, link.gale.com/apps/doc/A79351726/HRCA?u=mlin_oweb&sid=googleScholar&xid=ce7105c8.

The Academy of Orthopaedic Physical Therapy provides treatment recommendations and guidance to clinicians for the treatment of Patellofemoral Pain (PFP). While the Academy states that clinicians “may use tailored patellar taping in combination with exercise therapy to assist in immediate pain reduction, and to enhance outcomes of exercise therapy in the short term (4 weeks), they recommend against the prescription of patellofemoral knee orthoses (bracing) for patients with PFP. “Patellofemoral Pain—Clinical Practice Guidelines Linked to the International Classification of Functioning, Disability and Health From the Academy of Orthopaedic Physical Therapy of the American Physical Therapy Association,” J Orthop Sports Phys Ther. 2019; 49(9): CPG1-CPG95 (doi:10.2519/jospt.2019.0302), at page CPG3.

### SUMMARY OF THE DISCLOSURE

An objective of the present disclosure is to provide improved knee malpositioning mitigation devices and methods, that have aspects that are akin to treatments favored by clinicians such as patellar taping, for example, McConnell taping.

An objective of the present disclosure is to provide improved knee malpositioning mitigation devices and methods that allow for control and adjustment during intervention.

Another objective of the present disclosure is to provide patellar pain or malpositioning mitigation without interfering with clothing, and to provide device options that are easy to use, adjust, remove, and reuse.

One or more alternate or additional objectives may be served by the present disclosure, for example, as may be apparent by the following description. Embodiments of the disclosure include any apparatus, system, method, or article, or any one or more sub-parts or sub-combinations of such apparatus, system, method, or article, for example, as supported by the present disclosure.

Per one embodiment, apparatus is/are provided. A patellar anchor is provided that is dimensioned and configured to trace and apply sideways pressure to a perimeter edge of a human patella via portions of tissue covering and proximate to the same perimeter edge. At least one controlling member is provided which is configured to apply at least one counteracting force vector to the patella anchor to thereby cause or maintain the sideways pressure.

Per another embodiment, a method is provided. A patella anchor is positioned on a subject's knee in order to trace and apply sideways pressure to a perimeter edge of a human patella in a direction traversing a plane that lies in the center of the patella between the patella's anterior and posterior sides. The pressure is applied via portions of tissue covering and proximate to the same perimeter edge. At least one controlling member is positioned and fixably adjusted to apply at least one mitigating force vector to the patella anchor thereby causing or maintaining the sideways pressure. The patella anchor may be a rod member, which in some embodiments is firm and elastomeric.

### BRIEF DESCRIPTION OF THE DRAWINGS

Example embodiments will be described with reference to the following drawing figures in which:

FIG. 1 provides a perspective view of one embodiment of a patellar device of the present disclosure applied to exert a lateral bias on a subject's patella;

FIG. 2 provides an elevated view of an application side of the patellar device shown in FIG. 1;

FIG. 3 shows a representation of a skyline view of the patella in relation to the trochlear groove in the femur, in medial patellar tilt and lateral patellar shift positions;

FIG. 4 is a different view of the applied patellar device shown in FIG. 1.

FIG. 5 is a cross-sectional view of the applied patella device shown in FIG. 4, taken along the lines I-I;

FIG. 6 is a cross-sectional view of another embodiment of an applied patella device provided with an extended patella anchor;

FIG. 7 shows an embodiment of the patellar device applied to a subject's right leg, with attachment straps positioned and attached for lateral bias on the subject's patella;

FIG. 8 shows an embodiment of the patellar device applied to a subject's left leg, with the attachment straps positioned and attached for medial bias on the subject's patella;

FIG. 9 shows an embodiment of the patellar device applied to a subject's left leg for tibial bias on the subject's patella;

FIG. 10 shows an elevated front view of an embodiment of a patella device provided with an additional attachment strap positioned and attached for tilt mitigation;

FIG. 11 shows an elevated front view of an embodiment of a patella device provided with a push type controlling assembly;

FIG. 12 shows an elevated front view of an embodiment of a patella device provided with a surrounding patella anchor and a set of controlling members for setting and adjusting different and individually selectable bias force vectors; and

FIG. 13 shows an elevated side view of the device shown, for example, in FIG. 2;

FIGS. 14-17 show cross-sectional views in different embodiments of a supported patella anchor shown in FIG. 2, taken along the lines II-II;

FIG. 18 shows a cross-sectional view of an embodiment of an extended patella anchor shown in FIG. 10, taken along the lines III-III; and

FIGS. 19 and 20 show a cross-sectional view of a supported patella anchor, with example embodiments of a hot and cold gel compartment structure.

### DETAILED DESCRIPTION

Various apparatus and methods are provided, for example, as illustrated in the drawings. Referring generally to the figures, a patellar anchor 17 is provided that is dimensioned and configured to trace and apply sideways pressure to a perimeter edge of a human patella 20 via portions of tissue covering and proximate to the same perimeter edge. At least one controlling member (for example attachment straps 18, a sleeve 39, a web support 12) is provided which is configured to apply at least one counteracting force vector to the patella anchor to thereby cause the sideways pressure. In an embodiment of a method, a patella anchor is positioned on a subject's knee in order to trace and apply sideways pressure to a perimeter edge of a human patella in a direction traversing a plane that lies in the center of the patella between the patella's anterior and posterior sides. The pressure is applied via portions of tissue covering and proximate to the same perimeter edge. At least one controlling member is positioned and fixably adjusted to apply at least one mitigating force vector to the patella anchor thereby causing the sideways pressure. The patella anchor may be a rod member, which in some embodiments is firm and elastomeric.

The apparatus may be one or more devices and/or one or more articles. The apparatus may be customized to each patient.

The at least one controlling member is configured to generally cause biasing to be applied to the patella, and in certain embodiments, the at least one controlling member may also include certain tape elements as follows. Specifically, apparatus structure and/or method acts may be provided such that the at least one controlling member involves taping with a durable, high tensile strength tape. In certain embodiments, the durable high tensile strength tape may comprise a rigid strapping tape such as a zinc oxide tape, e.g., LEUKOTAPE brand tape. The taping may further involve taping with a protective layer beneath the durable high tensile strength tape, with a porous retention tape such as HYPAFIX tape. Protective layer taping allows for flexible wide area fixation for securing elements of the apparatus, is breathable, transversally stretchable, will not constrict, and is chemically inert to rubber and plastics.

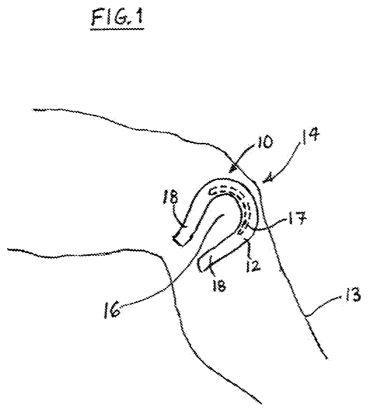

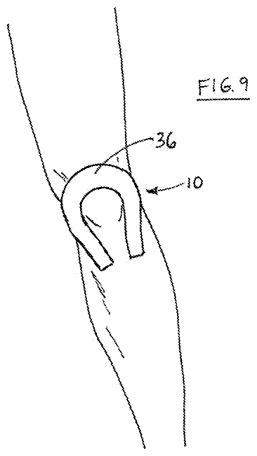

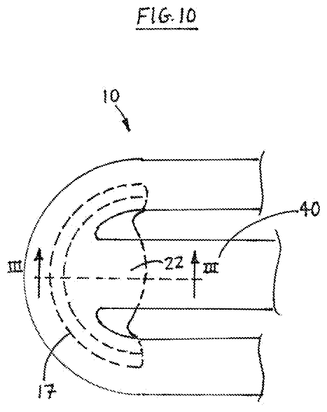

In alternative embodiments, or in addition, the apparatus structure and/or method acts may be provided such that the at least one controlling member involves taping with a frictional material such as adhesive silicone that allows adherence to human skin and gentle removal and repositioning. Referring, for example, to FIGS. 1, 2, 10, 11, and 12, one or more of the disclosed support members 44, 46 (FIG. 11), attachment straps 18, 40, and 52 (FIG. 12), and carrying web 12 may be made of adhesive silicone.

In alternative embodiments, or in addition, the apparatus structure and/or method acts may be provided such that the at least one controlling member involves taping with an elastic therapeutic kinesiology tape that allows adherence to human skin and gentle removal and repositioning.

In embodiments herein, the at least one controlling member acts on the patella anchor in order to control lateral tilt, glide and/or spin of the patella. The control may involve applying a mitigating bias force, by the controlling member pulling or pushing on the patella anchor. Each member may be a strap, a strip, a cord, or a rod, which includes a slender, flexible, elastomeric material. In one embodiment, the at least one controlling member comprise(s) one or more of flexible rods, straps, or cords, e.g., made of woven strands. The rod may be made of thread, yarn or an elongated web. The elastomeric material may include elastic therapeutic kinesiology tape with an adhesive surface.

The controlling member may be configured, for example, in the embodiment shown in FIG. 11 described more fully below, so as to push the patella anchor. In accordance with another embodiment, the controlling member is configured to pull the patella anchor. Lateral glide mitigation is configured by use of the apparatus (e.g., using one or more select integrated or separate devices forming a part thereof), wherein the counteracting force vector biases the patella toward the medial direction. This is done by the anchor being positioned along the outer lateral edge of the patella, and the at least one controlling member acting on the anchor to cause the medial direction counteracting force vector.

When the counteracting force vector biases the patella, in one embodiment, the anchor is shaped and positioned to contact at least two edges of the patella. Those edges may include an upper, a lower edge, and a lateral side edge of the patella.

The anchor may be controlled by the controlling member acting on the anchor to bias it in a rotational direction (counterclockwise) about an axis orthogonal to and at the center of the outer/forward facing (anterior) surface of the patella.

The patella anchor may be biased with a counteracting force vector acting on the patella toward a tilt mitigation direction, by the anchor being shaped and positioned to contact a central anterior surface of the patella, and by the controlling member acting on the anchor to create a bias force vector with one component in the medial direction and another component toward a posterior position of the leg.

The anchor may comprise a substantially C-shaped rod shaped like a sectionally halved part of a toroid. The C-shaped rod may have a rectangular cross section. In another embodiment, it has an oval cross section. In another embodiment, the C-shaped rod has a circular cross section. In another embodiment, the rod has a triangular cross section.

An inner side wall of the C-shaped member may be configured to be generally perpendicular to a surface traversing the patella's anterior.

In other embodiments, the anchor comprises an elastomeric material. The anchor may be made of adhesive silicone. The anchor may comprise a cord with woven and twisted composite rubber strands.

The flexible rod may be an elastomer. More specifically, the flexible rod may comprise rubber or synthetic rubber, a sponge, foam, adhesive silicone, or woven materials. The rod may be solid or hollow depending upon the embodiment.

The rod is configured so that it is sufficiently firm, resilient, and sufficiently adhesive at its surface so that it creates an amount of friction against the skin and soft tissue around the patella, in order to exert the proper force in the desired direction against the patella's edge, without being deformed too much. In one embodiment, the rod's firmness is between 20 and 96 Shore A in accordance with the ASTM D2240 standard. In another embodiment, the rod's firmness is between 50 and 70 Shore A.

Per another embodiment, the rod is comprised of polychloroprene (neoprene) which is configured to be sufficiently elastomeric to return to its initial shape, but also configured to be sufficiently firm in order to exert the appropriate force vector against the patella, that is, with a hardness in the range of 20-96 Shore A per one embodiment, and between 50 and 70 Shore A per another embodiment.

The counteracting force vector is applied to the patella, by means of the controlling member or members pulling or pushing depending upon the embodiment.

In one embodiment, the patella anchor is dimensioned and configured such that it extends past the edge of the patella to cover the front center (anterior) portion of the patella, so that when the controlling member(s) is/are adjusted, the front center of the patella is pulled in the desired direction in order to cause a tilt mitigation bias from the middle of the kneecap anterior to the side of the knee. When a lateral glide mitigation bias is applied, the controlling member or members are configured to pull the patella anchor toward a medial position.

When a rotational component is to be mitigated, one or more of the controlling members is configured to be attached to the patella anchor and pull or push the patella anchor in a rotational manner. For example, a portion of the controlling member or members may be configured to attach to a lower portion of the patella at a lateral side and to an upper position past on the medial side of the kneecap. This creates a counterclockwise bias in order to mitigate against a rotational component, otherwise referred to as spin.

In an embodiment, the patella anchor is made of fabric, and the fabric may be made from an elastane and foam combination of materials.

The patella anchor may be provided with an internal reinforcing portion with increased firmness throughout all or a substantial portion of its length, with an additional amount of rigidity, and shaped and configured to push against the edge of the patella when it is pulled or pushed by the controlling member or members.

In another embodiment, with the controlling member or members in a pulling configuration, they include, for example, one or more straps. That are extended and adhered around the knee. When performing a lateral mitigation, the straps are strapped around the knee in the medial direction. In one embodiment, one strap extends horizontally in the medial direction across and around the knee above the patella, and the other strap extends in the horizontal direction across and around a part of the knee below the patella.

When medial mitigation is applied, the upper strap is pulling the patella anchor in the opposite direction, i.e., the lateral direction, just above the patella and in the same direction just below the patella.

In accordance with another embodiment, a 360 degree wrap around sleeve is provided around the leg with a free opening not contacting or pushing against the patella at one side, while supporting the control member or members at the other side to cause the patella anchor to trace and come into firm contact with a side edge of the kneecap.

A carrying web may be provided, which is configured to carry the patella anchor. The carrying web in one embodiment, is in the form of a C shape. In another embodiment, it is in the form of an O shape. In one embodiment, the carrying web includes a sleeve configured to snugly fit around the leg at portions of the leg above, below, and around the knee. In some embodiments, the patella anchor is a C-shaped rod member, and has an outward facing side forming an outer curve of the C shape and an inner facing side forming an inner curve of the C shape. The inner facing side of the C shaped rod member is pressed in contact with the lateral side edge of the patella, when the patella anchor applies the sideways pressure to the lateral side edge in order to create a mitigating force acting on the patella.

In one embodiment, the pressure that is applied to the patella anchor is in a medial tracking direction parallel to the anterior surface of the patella. The lateral edge includes at least a lateral side edge of the patella. The perimeter edge in one embodiment is the lateral side edge of the patella. In another embodiment, the perimeter edge includes at least portions of top and bottom edges of the patella.

In the case where there is a mitigation of medial bias, the perimeter edge includes at least a medial side edge of the patella. More specifically, the perimeter edge may be the medial side edge of the patella. In another embodiment, the perimeter edge further includes at least portions of the top and bottom edges of the patella.

In another embodiment, there is mitigation against an upward bias by applying a mitigating force vector in the downward direction toward the tibia. In this case, the perimeter edge includes at least the top edge of the patella. It may be the top edge of the patella.

The method may include applying a patella anchor that further includes a firm pad that extends to a central anterior portion of the patella. Posterior pressure is applied on the anterior surface of the patella. The posterior pressure is applied via portions of tissue covering the central anterior portion of the patella.

Referring now to the drawings in greater detail, FIG. 1 shows a perspective view of one embodiment of a patellar device 10 of the present disclosure applied to exert a lateral bias on a subject's patella. Patellar device 10 is provided, which is on a bent knee of the subject. Specifically, patellar device 10 is on the subject's right leg 13 at a knee portion 14 of the leg on the outer skin surface. The applied patellar device is centered around the portion of the outer skin surface 16 located over the patella anterior.

The illustrated patellar device 10 includes a carrying web 12 which serves as a substrate or support for a patella anchor 17. The portion of carrying web 12 that is in contact with the skin of the subject is adhesive. The outer portion of carrying web 12, which is not in contact with the subject's skin, is not adhesive. Per one embodiment, carrying web 12 is made of silicone adhesive, which has adhesive properties when its surface is clean and comes into contact with human skin.

The illustrated patellar device 10 includes attachment straps 18, which, in this embodiment, are extensions of carrying web 12. Patellar device 10 as shown in FIG. 1 is applied in a way to exert a lateral bias on the patella in order to mitigate possible malpositioning or unwanted bias in the medial direction. It does this by positioning and then biasing the patella anchor 17, which includes a firm elastomeric rod in the illustrated embodiment. Patella anchor 17 is dimensioned and configured to trace and apply sideways pressure to a perimeter edge of the human patella in a direction traversing a central plane that lies between anterior and posterior sides of the patella. The pressure is applied via portions of tissue covering and proximate to the same perimeter edge.

The pressure in the illustrated embodiment shown in FIG. 1 is in the sideways direction—traversing the central plane of the patella. In this applied embodiment for mitigating unwanted medial bias, the sidewards direction is the lateral tracking direction.

Several example embodiments of the apparatus and methods of use are provided herein, for medial, lateral, and tibial bias per se. These embodiments are configured such that sideways pressure caused by the patella anchor causes a force vector acting on the patella that includes substantially only a component traversing the central plane of the patella. The force vector includes no material component providing a lifting (anterior) or compressing (posterior) force acting on the patella.

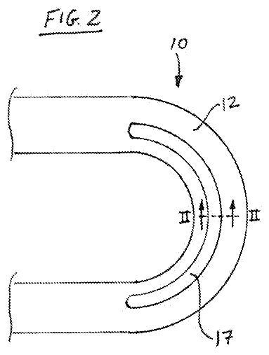

FIG. 2 provides an elevated partial view of an application side of the patellar device 10 shown in FIG. 1. “Application side” in this context means the side of the patellar device that is configured to be put in contact with the subject's skin. Carrying web 12 is shown, and a patella anchor 17 is fixedly placed on carrying web 12 in a curved fashion which follows the shape of an edge of the patella.

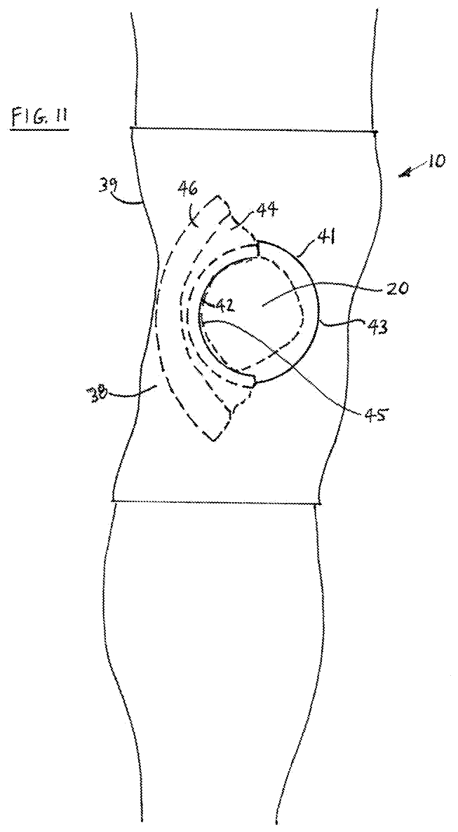

FIG. 13 is an elevated side view of the device 10 shown in FIG. 2, and shows the device with the knee/skin interface side facing upwards. As shown in FIG. 13, patella anchor 17 is adhered to and supported by web support 12. In an alternate embodiment, patella anchor 17 may be formed as an integral part of web support 12.

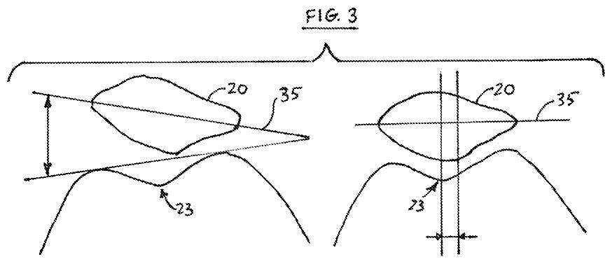

FIG. 3 provides a representation of a skyline view of the patella in relation to the trochlear groove 23 in the femur, and shows two (malpositioning) positions of the patella 20. The left view shows the patella in a medial tilt position, and the right view shows the patella in a lateral shift position. Line 35 represents the central plane of patella 20, that lies between anterior and posterior sides of patella 20.

FIG. 4 is a view of the applied patellar device 10 shown in FIG. 1 in relation to a solid-lined perimeter of patella 20, and it shows how patella anchor 17, specifically at an interfacing surface 19 of the patella anchor, is configured to trace and apply sideways pressure to the perimeter edge, in this case, the medial perimeter edge 26 of patella 20. This pressure is applied via portions of tissue, not specifically shown in FIG. 4, where that tissue covers and is proximate to the same perimeter edge 26. Although patella 20 is under skin and tissue, and therefore would normally be depicted with dotted lines if following drawing conventions, its perimeter is depicted with solid lines for purposes of more easily showing the perimeter in relation to the device.

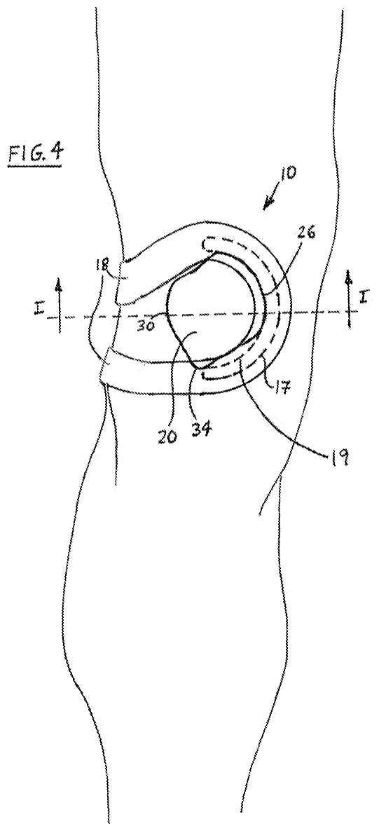

FIG. 5 provides a cross-sectional view of the applied patella device shown in FIG. 4 taken along the lines I-I. As shown in FIG. 5, interfacing surface 19 of patella anchor 17 is held up against the traced edge 26 of patella 20 via tissue 24 that is covering and proximate to perimeter edge 26. As shown in FIG. 5, which is a skyline view, patella 20 is positioned to align with a trochlear groove 23 in a femur 25 of a subject's leg.

Carrying web 12 has adhesive on its patella-side surface, which adheres to the skin surface 16, and accordingly holds the patella anchor 17 in position. Specifically, carrying web 12, serves as a controlling member combined with straps 18, such that, when it is properly placed, it applies at least one mitigating force vector to patella anchor 17. Patella anchor 17 thereby traces and applies a sidewards, in this case lateral, pressure to perimeter edge 26 of patella 20 in a direction traversing a central plane of the patella. The pressure is applied via portions of tissue 24 covering and proximate to the same perimeter edge 26.

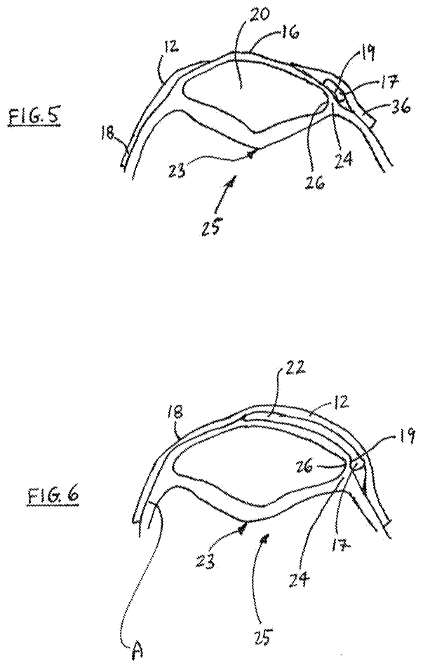

FIG. 6 shows a cross-sectional view of another embodiment of an applied patellar device 10 provided with an extended patella anchor 22. Specifically, patellar device 10 is illustrated in a skyline view in relation to a subject's femur 25, and patella 20 is positioned in trochlear groove 23. A portion of patella anchor 17, specifically, interface surface 19, is positioned to trace and apply pressure at the medial perimeter edge 26 of patella 20 and via the tissue 24 covering and proximate to the same perimeter edge 26 the patella.

Patella anchor 17 is configured in this embodiment to extend all the way up to the central anterior portion of the patella. Patella anchor 17 includes a firm pad 22 as an extended portion, which is dimensioned and configured to apply posterior pressure to a central anterior portion of the patella. When the device is properly put in place, as shown in FIG. 6, carrying web 12 is placed on and adhered to the skin covering each side of patella 20.

Referring back to FIG. 5, in operation, the first end 36 of carrying web 12 is adhered to the skin at one side of the knee, positioning patella anchor 17 for proper engagement with medial perimeter edge 26 of patella 20. The rest of carrying web 12 is adhered to the portions of the skin surrounding the perimeter of the patella 20, with extending attachment straps 18 alongside top and bottom sides of patella 20. Each of straps 18 is adhered to the skin on the opposite side of the knee at proper locations in order to apply the appropriate biasing force vector opposing the medial bias.

This same thing happens in the application of the device as shown in FIG. 6. The difference with FIG. 6 is that there is an extended portion 22 of patella that extends up to and just past the central anterior portion of the patella. In order to cause a medial tilt mitigation posterior force component, the attachment straps 18 may include a central strap positioned and adhesively secured in this embodiment to firm pad 22 at one position and to a position on and along the lateral side of the leg, at position A in this figure.

FIG. 7 shows, in another view, a configuration where the patellar device 10 is applied to the right leg of a subject in order to exert a lateral bias, to mitigate possible malpositioning in the medial direction. As described above, patellar device 10 is applied first at the curved side 36 at the medial side of the patella, taking care to ensure that the curvature of the interfacing surface 19 of patella anchor 17 (elements 19 and 17 are not visible in FIG. 7) is positioned to trace and push against medial perimeter edge 26 of patella 20. The remaining elongated portions of carrying web 12 are progressively adhered to the skin until attachment straps 18 are securely attached. This is done applying the appropriate amount of pressure so that once the entire carrying web 12 is adhesively attached, the patella anchor 17 continues to apply the appropriate amount of pressure with appropriate directional components, which provides a mitigating force vector that is desired against any potential medial biasing or malpositioning.

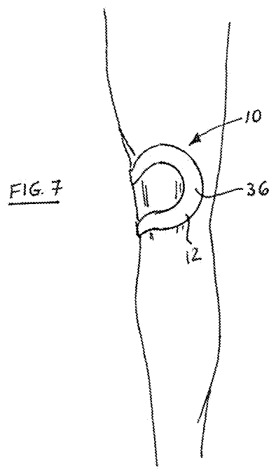

FIG. 8 provides an embodiment of the patellar device 10 applied to a subject's left leg with the attachment straps positioned and attached for a medial bias on the subject's patella. When the device is used in this manner, similar to the method described above as in relation to FIG. 7, the curved side 36 of carrying web 12 is first secured, positioning the patella anchor 17 so that it traces and applies the sidewards pressure to the perimeter edge on the lateral side (at the outer side of the leg, which is the left side of the left leg in this example). The sidewards pressure is in the medial tracking direction facing the inner side of the leg. The remaining extended portions of the carrying web 12 are adhesively applied to the skin at locations above and below the patella until the ends of attachment straps 18 are adhered along the inner side of the subject's left leg. This is done in a manner to ensure that the desired amount of pressure is placed on the perimeter edge, in this case the lateral perimeter edge, and remains an appropriate amount with desired directional components to create the desired medial bias to mitigate any possible malpositioning or undesired lateral bias.

The device may be recommended for use on the right knee to pull the patella in the medial direction, e.g., if there is pain in the right knee due after driving an automobile for a lengthy time. Such medial bias would mitigate valgus strain at the knee.

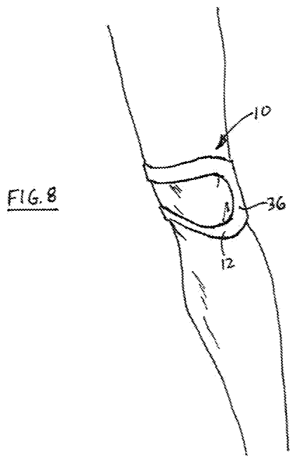

FIG. 9 shows the use of the device 10 applied to provide a bias toward the tibia. Here, the curved side 36 of carrying web 12 is first secured, positioning the patella anchor 17 so that it traces and applies downwards pressure (with no compression or lift components) to the upper perimeter edge of the patella. The downwards pressure is in the direction of the tibia or shin bone. The remaining extended portions of the carrying web 12 are adhesively applied to the skin at locations along the opposing sides of the patella until the ends of attachment straps 18 are adhered along sides of the tibia just below the knee. This is done in a manner to ensure that the desired amount of pressure placed on the upper perimeter edge remains an appropriate amount to create the desired tibial bias to mitigate any possible malpositioning or undesired upward bias.

This type of bias shown in FIG. 9 may be recommended, e.g., when one wants to mitigate a jumper's knee strain.

FIG. 10 shows an elevated view of the patellar device 10 in accordance with another embodiment. An additional strap 40 is connected to a central portion of carrying web 12, and it extends over and past elongated extended portion 22 of patella anchor 17. Strap 40 is adhered to extended portion 22 at or past the central anterior part of the knee cap, and then pulled with a biasing force so as to apply (to the central anterior part of the patella) a mitigating force with both sideways and posterior components.

FIG. 11 shows another embodiment of the patellar device configured so that the controlling member or members is/are configured to push the rod member of the patella anchor into a perimeter edge of the patella.

In the embodiment shown in FIG. 11, patellar device 10 includes a sleeve 39 configured to snugly fit around the leg at locations above and below the knee. Sleeve 39 includes a sleeve opening 41. Sleeve opening includes a push side edge 42 and an unobstructed side edge 43. Portions of the sleeve at or near push side edge 42 are configured to provide a sufficient amount of pressure into the leg and nearby portions of the knee in order to hold patella anchor 17 at a location so that it traces and applies pressure to the corresponding perimeter edge 45 of patella 20. In FIG. 11, patellar device 10 is applied to a patient's right leg, so the illustrated corresponding perimeter edge 45 is the lateral edge of patella 20.

A support web portion 38 of sleeve 39 (the left side in FIG. 11) is configured to support and cover at least patella anchor 17 and structures near and around patella 20 up to and including support side edge 42 of opening 41. As shown, in the illustrated embodiment, support web portion 38 provides support to a fortified support translation rod or member 44. Support web portion 38 further supports a base attachment strap 46, provided to hold fortified support translation rod or member 44 against patella anchor 17. In other embodiments (not shown), more than one base attachment straps may be provided to supplement and/or provide this hold.

In the illustrated embodiment, unobstructed side edge 43 of sleeve opening 41 is configured to provide freedom of movement to the patella and surrounding structures, and it therefore starts a substantial amount of distance away from the corresponding perimeter edge of the patella. In one example embodiment, between a half inch to one inch distance is provided from the corresponding medial edge of the patella to side edge 43.

The illustrated sleeve may be configured to slide on and off a user's leg, and/or to be attached by wrapping the sleeve and detachably attaching it behind or along a side of the leg with fasteners (not shown), e.g., hook and loop or another type of structure.

The sleeve embodiment shown in FIG. 11 comprises a push support configured to support a controlling member configured to push the rod member into the perimeter edge. In other embodiments (not shown), a sleeve could be configured to provide a pull type of controlling member that pulls the rod member of the patella anchor into the perimeter edge.

The push version of the controlling member or members, for example, as shown in FIG. 11, may be applied in order to provide a lateral bias in order to mitigate against unwanted medial bias or malpositioning. One may place the sleeve in the reverse direction on the same leg, or on the left leg, and thereby be able to configure patellar device 10 for medial bias in order to mitigate against lateral bias or malpositioning.

Alternatively, an embodiment may be provided that provides push structures on both sides of the knee where the desired push structure can be selectively engaged, while the other one is left unengaged. In addition, or alternatively, a bias attachment strap (or straps), fortified support translation rod or member (each not shown), and a corresponding patella anchor may be provided above the patella in order to push the patella down toward the tibia in order to mitigate, for example, jumper's knee.

FIG. 12 provides a view of an embodiment of a patellar device 10, with a sleeve 39 that includes a reinforced support portion 52 (which may be ring-shaped) provided to support a patella anchor 17. In this embodiment, support portion 52 is configured, for example, like the web support 12 in FIGS. 2 and 13, and is fixed to and securely supports anchor 17. Portion 52 has an opening 50 that, in this example embodiment, traces the inner perimeter of a ring-shaped patella anchor 17. The inner perimeter of patella anchor 17 is dimensioned and configured to trace and interface with the perimeter edges (upper, lower, lateral, and medial) of patella 20. While sleeve 39 is provided here, other embodiments may be provided, which have (in addition to or in lieu of a sleeve) one or more smaller supports (e.g., a web support) or no support for holding/carrying attachment straps and/or a patella anchor. An optional extended anchor portion 51 may be provided that covers the central anterior portion of the patella, generally for tilt mitigation.

A number of straps may be provided connected to the patella anchor structure 17 to allow for various types of adjusted and selectively applied mitigation biasing forces. For example, straps 52a and 52c may be configured to apply a pulling force so that the corresponding edges of patella anchor rod 17 at the other side trace and apply pressure to the lateral patellar edge. 30.

When applying the device shown in FIG. 12, it may be applied so that only one type of mitigating force is applied at a time in order to not apply unneeded and potentially counterproductive biasing forces to the patella.

If there is an indication that there is a need for a mitigation against medial malpositioning, if the device is on, for example, a subject's right leg, the other side pair of controlling members attachment straps 52f and 52d may be attached in a way to exert a pulling force, causing the corresponding edge of the patella anchor 17 (which may be, for example, a rod member) to trace and apply sideways pressure to medial perimeter edge 26.

It is possible that a given attachment strap or plural set of attachment straps may be individually or selectively provided. It's not necessary that all of the straps shown in the embodiment shown in FIG. 12 be actively connected to the device at one time.

The Illustrated patella anchor 17 does not necessarily have to be part of a sleeve. For example, it could be provided by itself, and then supported only by one strap or a set of attachment straps.

In one embodiment, a sleeve is provided and a push structure as shown in FIG. 11 may be utilized. That structure can be activated or deactivated depending upon what type of mitigating bias force is desired to act on the patella in a manner similar to that described with with the version of using pull type attachment straps.

In addition to a pair of attachment straps 52a and 52c, or in the alternative, a patellar tilt may be mitigated with the use of a central pull type attachment strap, for example, 52b to create a mitigating bias force against a patellar tilt. Alternatively, if the tilt is in the other direction, a central pull strap 52e may be activated and adjusted in order to provide the desired mitigating bias force vector. In the case of the use of the central straps, the patella anchor may be configured to include a firm pad that extends from the rod member at the perimeter edge up till the central anterior portion of the patella. The extended portion of the patella anchor may be dimension and configured to apply a posterior pressure to a central anterior portion of the patella in a direction toward a posterior position. In relation to the anterior surface of the patella, the posterior pressure is applied using the appropriate corresponding central strap, 52b or 52e, via portions of tissue covering the central anterior portion of the patella.

FIGS. 14-17 show cross-sectional views in different embodiments of a supported patella anchor shown in FIG. 2, taken along the lines II-II. FIG. 14 shows anchor rod 60 with a circular cross section, fixedly attached to base support 62 with, for example, an adhesive.

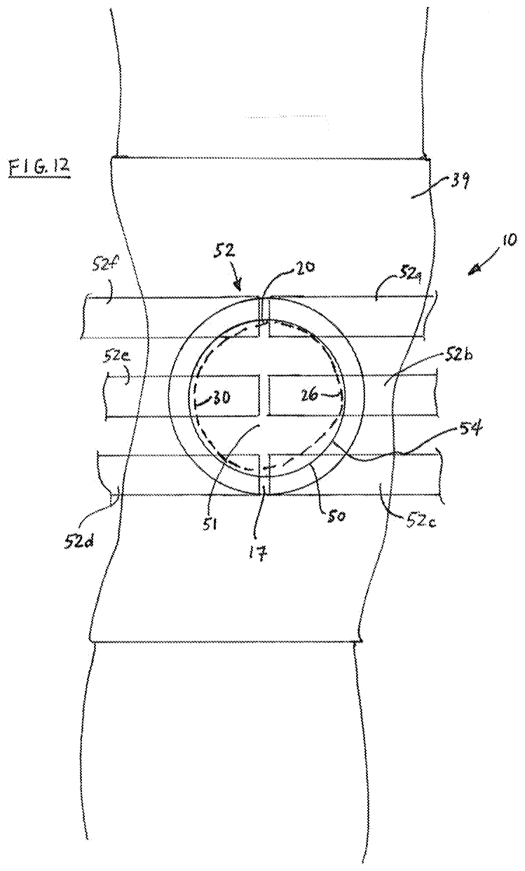

FIG. 15 shows anchor rod 60 formed integrally with base support 62, and having a rolling hill or oval shaped cross section.

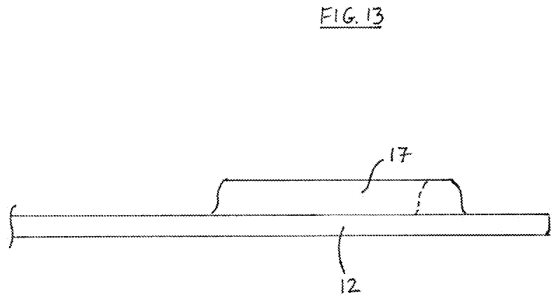

FIG. 16 shows anchor rod 60 with a triangular cross section with a ninety degree angle formed between a side facing the patella and base support 62, and a surface that is gradually tapered on the application side of the patella anchor.

FIG. 17 shows anchor rod 60 with a rectangular cross section formed integrally with base support 62.

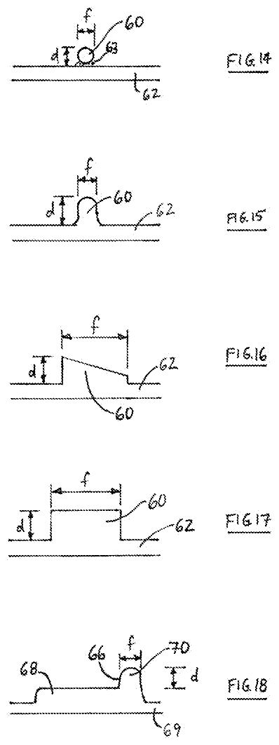

FIG. 18 is a cross-sectional view from FIG. 10, taken along the lines III-III, and shows a raised sideways anchor 70 for interfacing, at its perimeter interface 66, with the patella's perimeter edge, and an extended anchor 68 configured to contact and engage with the center of the patella's anterior side. Anchor structures 70 and 68 are integral with base support 69 in this embodiment.

While in the embodiment shown in FIG. 14, the patella rod 60 is attached to its base 62 with a permanent adhesive, in each of the embodiments shown in FIGS. 14-20, the patella rod 60 may be (permanently or removably and adjustable) attached to its base 62 with an adhesive or some other glue or fastener on the one hand, or may be integral with its base 62 on the other. The dimensions of the patella anchor structures shown in each of FIGS. 14-18, for example, are delineated with a height d and a length f. In the embodiments, d has a value between 2 mm and 2 cm, and f is between 1 and 4 times d. Per one example embodiment, d is between 7 and 13 mm. These are example embodiments, and do not preclude other embodiments with different dimensions d and f.

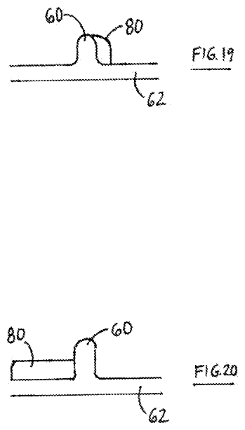

FIGS. 19 and 20 show a cross-sectional view of a supported patella anchor 60, with a hot and cold gel compartment structure 80. In FIG. 19, a compartment 80 is provided behind patella anchor 60 on a side opposite to the side that traces and interfaces with the patella edge. In FIG. 20, a compartment 80 is provided in front of patella anchor on the side that traces and interfaces with the patella edge. In this embodiment, gel compartment 80 has a lower height than patella anchor 60, and is configured to be against the skin covering the anterior of the patella at a location at or near the patella's perimeter.

In the embodiments shown in FIGS. 19 and 20, one or more gel compartments may be provided, e.g., forming two or more gel cells along or near the patella anchor. The compartments may be made of a urethane material. Depending on the embodiment, each compartment may contain a hot and cold gel, e.g., including sodium polyacrylate, super absorbent polymerSAP, hydroxyethyl cellulose, and vinyl-coated silica gel. U.S. Pat. No. 3,780,537 provides further information concerning the provision of hot and cold gel compartments, and discloses various features that may be provided in accordance with one or more embodiments disclosed herein employing hot and cold gel compartments.

In one or more of the above embodiments, including one or more of those depicted in FIGS. 14-18, a central portion of the anchor may be configured to have a reinforcing rod extending throughout all or a substantial portion of the length of the anchor. Such a reinforcing portion may be a firmer elastomeric material than the rest of the anchor.

The claims as originally presented and as they may be amended, encompass variations, alternatives, modifications, improvements, equivalents and substantial equivalents of the embodiments and teachings disclosed herein, including those that are presently unforeseen or unappreciated, and that, for example, may arise from applicants/patentees and others.

What is claimed is:

1. Apparatus comprising:
a patella anchor dimensioned and configured to trace and apply sideways pressure to a perimeter edge of a human patella in a direction traversing a central plane of the patella that lies between the patella's anterior and posterior sides, the pressure being applied via portions of tissue covering and proximate to the same perimeter edge; and
at least one controlling member configured to apply at least one mitigating force vector to the patella anchor which thereby causes or maintains the sideways pressure.

2. The apparatus according to claim 1, wherein the patella anchor comprises a firm elastomeric rod member.

3. The apparatus according to claim 1, wherein the sideways pressure is in a medial, lateral, top, or bottom tracking direction.

4. The apparatus according to claim 1, wherein the patella anchor further includes a firm pad dimensioned and configured to apply posterior pressure to a central anterior portion of the patella, the posterior pressure being applied via portions of tissue covering the central anterior portion of the patella.

5. The apparatus according to claim 1, further comprising a carrying web configured to carry the patella anchor.

6. The apparatus according to claim 5, wherein the carrying web is in the form of a C shape.

7. The apparatus according to claim 5, wherein the carrying web comprises a sleeve configured to snugly fit around the leg at portions of the leg above, below, and around the knee.

8. The apparatus according to claim 2, wherein the rod member is in a shape of a C, the C-shaped rod member having an outward facing side, forming an outer curve of the C shape and an inner facing side forming an inner curve of the C shape, the inner facing side of the C-shaped rod member being pressed in contact with the lateral side edge of the patella when the patella anchor applies the sideways pressure to the lateral side edge in order to create a mitigating force acting on the patella.

9. The apparatus according to claim 1, wherein the patella anchor comprises a rod member formed in a shape in order to interface with and apply pressure to the patella's perimeter edge.

10. The apparatus according to claim 7, wherein the sleeve comprises a push support configured to support a controlling member configured to push the rod member into the perimeter edge.

11. The apparatus according to claim 7, wherein the sleeve comprises a pull support configured to support a controlling member configured to pull the rod member into the perimeter edge.

12. The apparatus according to claim 1, wherein the at least one controlling member comprises two attachment straps, each with adhesive and fixed to the patella anchor at one end and configured to be removably adhered to the skin of the leg at an end opposite the one end.

13. The apparatus according to claim 12, wherein the attachment straps are positioned and adhered in order to apply to the patella anchor a mitigating force vector in a direction causing the patella anchor to apply the sideways pressure, wherein the side edge of the patella is the lateral, medial, top, or bottom side edge of the patella.

14. A method comprising:
a patella anchor dimensioned and configured to trace and apply sideways pressure to a perimeter edge of a human patella in a direction traversing a central plane of the patella that lies between the patella's anterior and posterior sides, the pressure being applied via portions of tissue covering and proximate to the same perimeter edge; and
at least one controlling member configured to apply at least one mitigating force vector to the patella anchor which thereby causes or maintains the sideways pressure.

15. The method according to claim 14, wherein the sideways pressure is in a medial, lateral, top, or bottom tracking direction.

16. The method according to claim 14, further comprising carrying the patella anchor with a carrying web.

17. The method according to claim 16, wherein the carrying web comprises a sleeve configured to snugly fit around the leg at portions of the leg above, below, and around the knee, wherein the sleeve comprises a push support configured to support a controlling member configured to push the rod member into the perimeter edge, wherein the sleeve comprises a pull support configured to support a controlling member configured to pull the rod member into the perimeter edge.

18. The method according to claim 16, wherein the carrying web comprises a sleeve configured to snugly fit around the leg at portions of the leg above, below, and around the knee, wherein the sleeve comprises a pull support configured to support a controlling member configured to pull the rod member into the perimeter edge.

19. The method according to claim 14, wherein the at least one controlling member comprises two attachment straps, each with adhesive fixed to the patella anchor at one end and configured to be removably adhered to the skin of the leg at an end opposite the one end.

20. The method according to claim 19, wherein the attachment straps are positioned and adhered in order to apply to the patella anchor a mitigating force vector in a direction causing the patella anchor to apply the sideways pressure, wherein the side edge of the patella is the lateral, medial, top, or bottom side edge of the patella.
